(19) United States
(12) Patent Application Publication
(10) Pub. No.: US 2021/0366604 A1
(43) Pub. Date: Nov. 25, 2021

(54) SUPPLYING PATIENTS WITH MEDICAL EQUIPMENT FOR REMOTE MEDICAL INTERACTION WITH HEALTHCARE PROVIDERS

(71) Applicant: Rescue Box, Inc., Southold, NY (US)

(72) Inventors: Steven Templeton, Greenport, NY (US); Michelle Pelletier, Greenport, NY (US)

(21) Appl. No.: 17/115,213

(22) Filed: Dec. 8, 2020

Related U.S. Application Data
(60) Provisional application No. 63/026,884, filed on May 19, 2020.

(51) Int. Cl. G16H 40/67; A61J 1/00

(52) U.S. Cl. CPC G16H 40/67; A61J 2205/10; A61J 1/00

(57) ABSTRACT

A method for supplying patients with medical equipment for remote medical interaction with healthcare providers is provided. The method includes storing a medical supply container within a storage assembly. The medical supply container including medical equipment. The method further includes receiving a first input through a user interface in communication with the storage assembly. The method also includes selectively providing access to the medical supply container based on the input received through the user interface to enable the medical supply container to be removed from the storage assembly for the use, repair, replacement, and/or cleaning of the medical supply container and/or the medical equipment included therein.

### CROSS-REFERENCE TO RELATED APPLICATIONS

This application claims the benefit of and priority to U.S. Provisional Patent Application Ser. No. 63/026,884, filed on May 19, 2020, the entire contents of which are incorporated by reference herein.

### TECHNICAL FIELD

This disclosure relates to telemedicine and, more particularly, to systems and methods for supplying patients with medical equipment for remote medical interaction with healthcare providers.

### BACKGROUND

Telemedicine is the use of telecommunication, satellite communication and information technology to provide clinical health care from a distance. It has been used to overcome distance barriers and to improve access to medical services that would often not be consistently available in distant rural communities. It is also used to save lives in critical care and emergency situations. Although there were distant precursors to telemedicine, it is essentially a product of 20th-century telecommunication and information technologies. These technologies enable communications between patient and medical staff with both convenience and fidelity, as well as the transmission of medical, imaging, and health informatics data from one site to another.

Early forms of telemedicine achieved with telephone and radio have been supplemented with videotelephony, advanced diagnostic methods supported by distributed client/server applications, and additionally with telemedical devices to support in-home care.

### SUMMARY

According to one aspect, a method for supplying patients with medical equipment for remote medical interaction with healthcare providers is provided. The method includes storing a medical supply container within a storage assembly. The medical supply container includes medical equipment. The method further includes receiving a first input through a user interface in communication with the storage assembly. The method also includes selectively providing access to the medical supply container based on the input received through the user interface to enable the medical supply container to be removed from the storage assembly for the use, repair, replacement, and/or cleaning of the medical supply container and/or the medical equipment included therein.

In an aspect of the present disclosure, the method may further include selectively receiving the medical supply container in the storage assembly after the medical supply container is removed from the storage assembly

In another aspect of the present disclosure, the method may further include preventing access to the medical supply container when the medical supply container is supported in the storage assembly unless the first input includes predetermined information.

In yet another aspect of the present disclosure, the method may further include preventing access to a storage area in the storage assembly that supports the medical supply container after the medical supply container is removed from the storage area.

In a further aspect of the present disclosure, the method may further include receiving a second input through the user interface when the medical supply container is received in the storage assembly, the second input indicating at least one of the following: that the medical supply container was returned, the medical equipment was returned, that the medical supply container was opened, the medical equipment was opened, that the medical supply container was not opened, the medical equipment was not opened, that the medical supply container was used, the medical equipment was used, that the medical supply container requires cleaning, the medical equipment requires cleaning, that the medical supply container requires replacement, the medical equipment requires replacement, that the medical supply container is ready for use, the medical equipment is ready for use, the medical equipment requires repair, and/or the medical supply container requires repair.

In yet a further aspect of the present disclosure, the method may further include supplying the medical supply container with a blood pressure cuff, a pulse oximeter, an electronic stethoscope, a smart device, a thermometer, and an otoscope.

In an aspect of the present disclosure, the smart device may include a smartphone or a tablet.

In an aspect of the present disclosure, the method may further include receiving a code through the user interface to identify the medical supply container and/or the medical equipment thereof.

In another aspect of the present disclosure, the method may further include determining a status of the medical supply container and/or the medical equipment thereof based on the second input.

In yet another aspect of the present disclosure, the method may further include storing the status on the storage device or communicating the status to a server and/or users in communication with the server.

In a further aspect of the present disclosure, the method may further include maintaining the storage assembly in a pharmacy, in a building having a pharmacy, in a curtilage of the pharmacy, or in a curtilage of the building with the pharmacy.

In yet a further aspect of the present disclosure, the method may further include storing a plurality of medical supply containers in the storage assembly.

In accordance with aspects of the disclosure, a system for supplying patients with medical equipment for remote medical interaction with healthcare providers is provided. The system includes a storage assembly that supports at least one medical supply container, a processor, and a memory. The memory has instructions stored thereon, which when executed by the processor, cause the system to: receive a first request from a user interface for the at least one medical supply container, determine if the first request meets a first predetermined criteria, and provide access to the at least one medical supply container if the first predetermined criteria is met.

In an aspect of the present disclosure, the instructions, when executed by the processor, may cause the system to: receive a second request from the user interface to return the at least one medical supply container, determine if the second request meets second predetermined criteria, and if the second predetermined criteria is met, provides access to a storage area within the storage assembly to return the at least one medical supply container.

In another aspect of the present disclosure, the instructions, when executed by the processor, may cause the system to prevent access to the at least one medical supply container if the first predetermined criteria is not met.

In yet another aspect of the present disclosure, the system may further include a server in electrical communication with the storage assembly, the medical supply container, and/or the medical equipment.

In a further aspect of the present disclosure, the server may be configured to communicate with a user.

In yet a further aspect of the present disclosure, the user may include a patient, a healthcare provider, a service provider, a vendor, a pharmacy, and/or a hospital.

In accordance with aspects of the disclosure, a system for supplying patients with medical equipment for remote medical interaction with healthcare providers is provided. The system includes a plurality of storage assemblies supporting a plurality of medical supply containers, a server in electrical communication with at least one of the plurality of storage assemblies and/or the plurality of medical supply containers, the server configured to receive a request through a user interface for temporarily providing access to a first medical supply container of the plurality of medical supply containers.

In an aspect of the present disclosure, the server may be configured to receive a request through the user interface for returning the medical supply container to one of the plurality of storage assemblies.

In another aspect of the present disclosure, the server may be configured to communicate with a user.

In yet another aspect of the present disclosure, the user may include a patient, a healthcare provider, a service provider, a vendor, a pharmacy, and/or a hospital.

In a further aspect of the present disclosure, healthcare providers may include doctors, dentists, nurses, physician assistants, therapists, pharmacists, trainers, nutritionists, and/or other healthcare personnel.

In yet a further aspect of the present disclosure, the service provider may include a repair service provider, a cleaning service provider, and/or an equipment supplier.

In an aspect of the present disclosure, the server may include input and/or output modules for transmitting and receiving input and/or output signals between at least one of components of the system or users of the system.

Other aspects, features, and advantages will be apparent from the description, the drawings, and the claims that follow.

### BRIEF DESCRIPTION OF DRAWINGS

The accompanying drawings, which are incorporated in and constitute a part of this specification, illustrate aspects of the disclosure and, together with a general description of the disclosure given above and the detailed description given below, serve to explain the principles of this disclosure, wherein:

FIGS. 1-3 are views of interface apparatus of systems for supplying patients with medical equipment for remote medical interaction with medical personnel in accordance with principles of this disclosure;

FIG. 4 is a perspective view of the medical supply container in an open position with medical components thereof shown positioned in the medical supply container;

FIG. 5 is a block diagram of a controller for the system of FIG. 1, in accordance with the present disclosure;

FIG. 6 is a flow diagram for a method for supplying patients with medical equipment for remote medical interaction with healthcare providers, in accordance with the disclosure;

FIGS. 7A-7F are progressive views illustrating a medical supply container of the system for supplying patients with medical equipment for remote medical interaction with medical personnel being retrieved from a storage unit of the system; and

FIG. 8 is a view illustrating the system communicating with a computing device of a patient.

### DETAILED DESCRIPTION

Aspects of the disclosed systems and methods are described in detail with reference to the drawings, in which like reference numerals designate identical or corresponding elements in each of the several views. In the following description, well-known functions or constructions are not described in detail to avoid obscuring the present disclosure in unnecessary detail. As commonly known, the term “clinician” refers to a doctor, a nurse, clinician assistant or any other care provider and may include support personnel. In addition, directional terms such as front, rear, upper, lower, top, bottom, and the like are used simply for convenience of description and are not intended to limit the disclosure attached hereto.

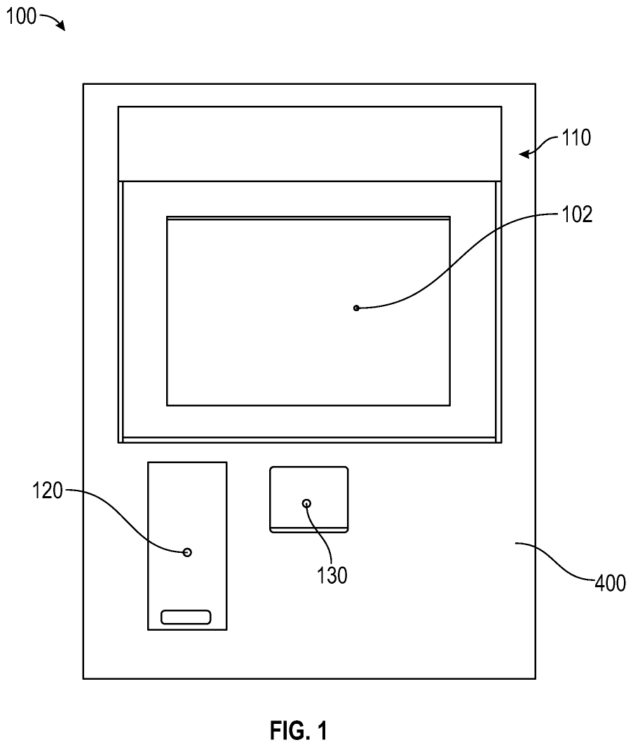

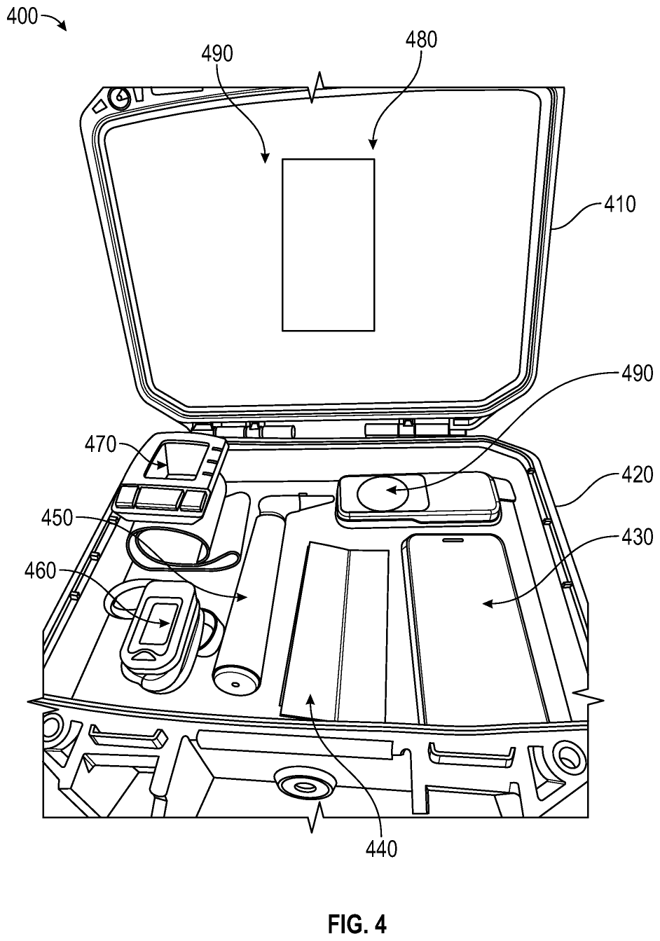

With reference to FIGS. 1-4, systems of this disclosure for helping supply patients with medical equipment for remote medical interaction with healthcare providers (e.g., telemedicine) are shown. The disclosed systems include storage assemblies 100, 200 (e.g., kiosks, storage lockers, or smart storage lockers) that may be smart. Other systems may simply include pick-up and drop-off stations at pharmacies or other retail environments in which tools/equipment scanned in and out by an employee, e.g., pharmacist. The disclosed storage assemblies 100, 200 support any number of medical supply containers 400 (FIG. 4) or boxes that selectively secure or lock medical supplies therein. These medical supplies include equipment that help to facilitate remote medical interaction with healthcare providers.

Further, as used herein, the term “provider,” “business,” “vendor,” “supplier,” and like terms are used to indicate an entity that works in concert with the storage centers, storage assembly, medical container, and/or medical equipment owner(s) or operators to provide users with the medical equipment for effectuating telemedicine.

With reference to FIG. 4, a medical supply container 400 is shown. The medical supply container 400 generally includes a lid 410 and a base 420 configured to hold medical supplies and/or equipment. For instance, the medical supply containers 400 may include the medical supplies and/or equipment (sometimes referred to as medical kit) such as a blood pressure cuff 470 (e.g., electrical), a pulse oximeter 460, an electronic stethoscope 490 (e.g., Bluetooth) and ECG, a smart device 430 (e.g., smartphone or tablet), a thermometer 440 (e.g., disposable thermometer strip), and an otoscope 450. In aspects, the medical kit may be modular and/or easily customizable at the point of pick up. For example, the medical kit may be automatically configurable by picking from an array of known kit components and assembling for pick-up at a kiosk.

The electronic stethoscope 490 can be an EKO Bluetooth stethoscope and ECG that allows a provider to listen to heart, lungs, abdomen, and carotid arteries and provides ECG for provider to review. The otoscope 450 can be a wireless otoscope ear camera that allows the provider to see ears, eyes, nose, mouth, and teeth. The blood pressure cuff 470 can be a blood pressure wrist cuff that provides a patient's blood pressure and heart rate. The pulse oximeter 460 can be a finger pulse oximeter that provides oxygen saturation in blood and heart rate. The thermometer 440 can be thermometer strip or tempdot that is a disposable thermometers that provides body temperature. The smart phone 430 connects all contents (e.g., test/measurement results, pictures, communication, etc.) and allows for telemedicine. The smart phone will include HIPPA compliant software. In aspects, the medical supply container may include a computing device coupled thereto including any suitable software and/or hardware (e.g., a touch screen, processor, memory, sensors, camera, display, keyboard, mouse, chips, modules, communications modules, input/output, etc.) similar to that disclosed in U.S. Pat. No. 10,617,522, the entire contents of which are incorporated by reference herein. The medical supply containers 400 can include disposable bags and/or sterilization pouches for supporting the medical supply container and/or the supplies/equipment therein. Other diagnostic tools/equipment may be included in the medical supplies or equipment, e.g., blood sampling tools/equipment, saliva sampling tools/equipment, urine of fecal matter sampling tools/equipment, etc.

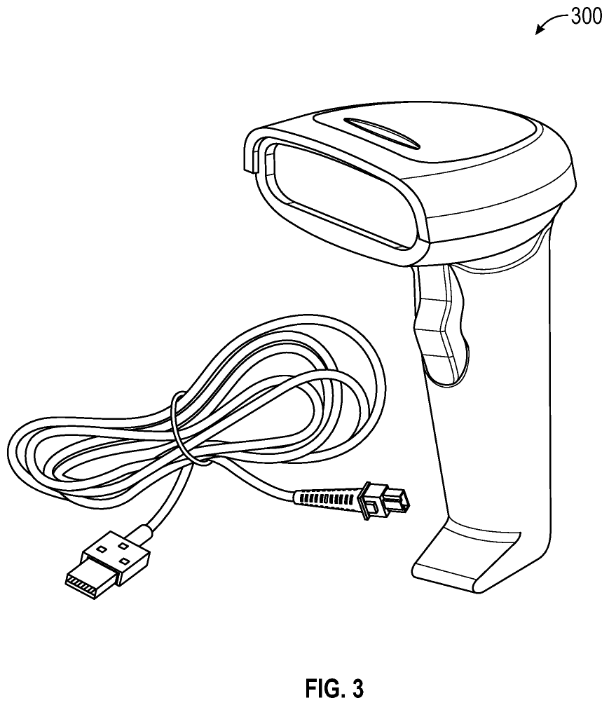

When a user, such as a patient, would like a medical exam, for example, the patient or other third party retrieves the medical supply container 400 from one of the storage assemblies 100, 200 or goes to a designated pick-up area. The storage assemblies 100, 200 include or are operatively coupled to a user interface 110, 210 (e.g., a touch screen 102, 202, scanner 130, 230, 300, a web-based platform, etc.) that enables someone to access the medical supply containers 400 supported therein upon inputting appropriate information (e.g., a name, a code, ID, credit card, phone number, address, and/or the like). A user may, for example, be prescribed a medical kit via a clinician or simply access a website and answer a few questions which prompt the user to collect the data (via the medical kit) for a clinician review. The user then simply picks up the medical kit that includes the necessary medical diagnostic tools at a local kiosk and returns the medical kits when completed. The patient may simply collect medical data (e.g., blood samples, EKG, urine samples, swabs, etc.) and return the samples to the kiosk (or another place of deposit) for further medical review. The test results are then sent to the clinician in any way known in the industry. In other instances, the medical kit may include tools/equipment that the patient uses to provide live feedback to the clinician during a phone call or televisit, which allows the clinician to review the patient during the call. The storage assemblies 100, 200 may include a scanner 300 (FIG. 3), which may be, for example, configured to scan bar codes to track the individual medical kits, and which may be hand-held and/or built-in to storage assemblies 100,200.

In aspects, such storage assemblies 100, 200 can be located in any suitable location such as by or in a pharmacy, a grocery store, a department store, a train station, airport, office building, government building, park, etc. For instance, a user may pick up the medical kit, and upon returning a medical supply container 400 to storage assembly in a pharmacy, the clinician may have already prescribed various medications that be ready for pick-up simultaneously with a return of the medical supply container 400. Moreover, the storage assemblies 100, 200, can be roving storage assemblies 100, 200, or robots that store, deliver, and/or pick-up such medical supply containers 400 and/or medical equipment. Such roving storage assemblies can be any suitable autonomous or semi-autonomous vehicles (e.g., drones, cars, trucks, aircraft, ships, trains, robots, motorbikes, etc.) that can communicate with each other, one or more servers/controllers, customers, vendors, and/or other service providers (e.g., repair shops), for example. The storage assemblies may be partly or entirely replaceable units that communicate with a storage center for replacement when the storage assemblies are running low. In other aspects, the storage assemblies are modular, and each individual storage unit (form an array of storage units) may be replaceable. In this instance, each individual storage unit may be customized for an individual patient at the main distribution center and loaded into the array of storage units for pick up by the patient via some identification and verification system.

In still other aspects, the array of individual storage units may be customizable on-site for particular patient needs. For example, a large array of storage units may include a storage center or warehouse of different diagnostic equipment/tools that may be automatically picked and assembled into kits and then placed into a storage unit for patient pick-up upon identification and verification. A clinician may simply enter what tests or tools/equipment are needed in a particular instance, and the storage center picks from the warehouse and assembles kits for patient pick-up. The storage center may also be modularized such that the various tools/equipment or tests may be simply loaded into the storage center for subsequent assembly.

These storage assemblies 100, 200 (or storage centers which is an array of storage assemblies 100, 200) can include a server or controller, which may be locally coupled thereto and/or remote therefrom (e.g., cloud-based), that can communicate with the users such as clinicians, patients, trainers, etc. and can enable selective access to these medical supply boxes for temporary and/or permanent use thereof (e.g., telemedicine). For example, a patient can take the medical supply container 400 home and use the medical equipment within the medical supply container 400 (e.g., temperature, oxygen levels, EKG, blood pressure, pictures) and send the same to a clinician such as via the smartphone included within the medical supply container 400 so the clinician can remotely monitor and/or diagnose the patient either virtually or within a convenient time frame thereafter so that the patient does not have to go to the clinician, hospital, and/or office in person. The storage centers, storage assemblies 100, 200, medical supply containers 400, and/or medical equipment thereof can be integrated into a platform (e.g., web-based) so that a network of information about patients, clinicians, service providers, the storage centers, the storage assemblies 100, 200, medical supply containers 400, and/or medical equipment, etc. can be exchanged, stored, transmitted, updated, etc. amongst the network to help, for example, identify status, appointments, medical information, repairs, replacements, locations, addresses, etc. of provider, component, user, patient, supplier, etc. in the network.

Advantageously, this helps promote safe social distancing, reduces unnecessary travel, waiting, and enables sick individuals to get medical attention right from their own home. This service can be provided 24/7 and allows online or kiosk purchasing/renting of such containers and/or equipment.

Once the user is done, the medical supply container 400, including some or all of the reusable and/or disposable equipment, can be returned to one of the storage assemblies 100, 200 for replacing, cleaning, repairing the equipment therein so that the medical supply container 400 and the medical equipment therein can be used by another. The medical container 400 and/or equipment therein can be scanned, for example, to alert a supplier, patient, or service person (e.g., via the server) that the medical supply box (and its contents) has been returned so that any repair, replacing, and/or cleaning can be effectuated by the appropriate service provider and/or that a receipt or prescription can be provided. Such service providers can come to the storage assemblies 100, 200 at regular intervals and/or upon an alert from the server to retrieve used medical supply containers 400 (and/or equipment thereof) for effectuating the necessary repair, replace, and/or cleaning services thereto.

In aspects, the system, storage unit, or storage center may be configured to automatically notify the clinician or services associated with the clinician once the medical kit is picked up and/or returned. Moreover, the system, storage unit, or storage center may cooperate with the clinician's scheduling software or platform, e.g., ZocDocs™, such that the software confirms the patient's appointment (or a step in confirming the appointment) with the clinician once the medical kit is picked up. Notifications may also be coordinated with the clinician's scheduling software or platform to alert or remind the patient to pick up the medical kit, or the medical kit is ready for pick-up.

In aspects, the clinician may cooperate with a software program or platform to customize the medical kit for a particular patient. Once the various medical tools/equipment and tests are selected by the clinician, the patient may be queried to select a location for pick-up for the customized medical kit via locality or zip code. The software program or platform may then either query various locations that have the customized kits already available based on the clinician's selections, query locations that can readily assemble the various kits for assembly based on the current inventory of tools/equipment and tests, or query locations that can receive the assembled kits from another location or have the various medical equipment/tools that may be assembled once shipped to the storage facility or center (e.g., the storage center can readily assemble kits once all of the tools/equipment and tests are received).

In aspects, the storage facility or center may include a wide array of standardized medical kits that are immediately available for pick-up. In instances, various tools/equipment may be included in a medical kit that is not intended to be utilized by a particular patient, which makes the medical kit more versatile and enable a standard kit to be used for a larger percentage of patients eliminating the need to customize the medical kits.

In aspects, the patient may pre-empt the clinician's appointment and pick up a medical kit and be ready for the appointment. This may warrant a more simplistic kit that the patient may pick up without a prescription.

In other aspects, the medical kit may include tools/equipment and tests that simply do not require a prescription and the patient may opt to take certain tests or use certain equipment prior to contacting a clinician. For example, the patient may pick up a standard kit that included an automated glucose monitoring system. When used, the glucose monitoring system may prompt the person that there is a need to contact a medical professional based on a detected abnormality. Other systems may automatically notify a designated/selected clinician that a medical kit has been picked up simply alerting the clinician that a patient has picked up a medical kit, and there may be a concern. The clinician's software program or platform may monitor these situations and may send prompts or alerts to either the patient or clinician regarding usage, compliance, or results.

In aspects, the medical kits may be tied into a software program or platform that alerts the clinician when a medical kit has been picked up, and a patient may need clinician support. The medical kit may also prompt the patient for reminders either prior to use and that the medical kit is awaiting use or during use that a clinician needs to be contacted immediately, or after use that the clinician is aware that the medical kit has been picked up use and is waiting for the patient to confirm an appointment.

More specifically, the medical kit may be equipped with timing software that prompts the patient to use the medical kit or to return the medical kit after use or because it has not been used. The software may be tied into the clinician's software program or platform to keep the clinician up to date of these activities or lack thereof.

The medical kit may include software that automatically alerts the clinician during use if there is an abnormal condition or a need to contact the patient, EMT or emergency contact. As mentioned above, after use, the medical kit may coordinate with the clinician's software program or platform to book a follow-up appointment, as necessary.

The medical kit may include software that automatically alerts the clinician after the kits has been used and prompts the storage facility to expect the return of the equipment or tests (e.g., swab tests) are to be expected for transfer to a testing facility. Reminders may be automatically configured once the patient is finished with the medical kit. Prescriptions may be filled and ready for pick-up as needed at the same or another site.

In aspects, the storage center may include protocols for ensuring that each medical kit and the contents therein are sterilized or sterilizable prior to pick up and after drop-off. Some protocols may include different drop-off and pick facilities or drop off and pick up locations at the same medical facility. Other protocols may include equipment/tools being dropped off at a storage center and any tests being dropped off at a separate location or center. Proper bio-containment bags may be included as part of the medical kit. Other protocols may include providing each storage locker for containing the medical kit to include various safety protocols to ensure sterilization, e.g., UV lighting (e.g., germicidal UV-C light), temperature control, active sterilant dispersion, etc.

As mentioned above, the patient or user interface may be a downloadable application or program that allows a patient to query the availability of a medical kit, location for pick-up and return of medical kit, query or allow input for various tests and tools needed, communicate with the medical kit, communicate with the clinician or clinician software or platform (e.g., scheduling, patient input or data, results), communicate with the tools/equipment in the medical kit, and/or receive reminders, alerts or notifications as needed. For example, the downloadable application may operate on a user's personal computer or mobile device.

In aspects, the entire system may include a large distribution system that supplies and collects medical kits all over the country or region-by-region. All of these systems are configured to communicate with one another and may be automized. The distribution system may be configured to handle standardized medical kits for distribution and collection or customizable medical kits for assembly anywhere down the distribution line of succession.

For example, a large regional distribution warehouse may receive standardized medical kits for distribution. The large regional warehouse may maintain an inventory of medical kits/supplies and distribute the medical kits to one or more intermediate facilities based on use and anticipated need in a given state. From there, the state may maintain its own inventory and distribute the medical kits to one or more smaller facilities based on use and anticipated need in a given state county, for example. The county may maintain its own inventory and distribute the medical kits to one or more smaller facilities based on use and anticipated need in a given town. Collection may work similarly feeding back to the regional distribution center for sterilization and restocking and re-use of a medical kit as needed. Certain tests contained in the medical kit may be removed from the kit for forwarding to a testing facility at any point during the distribution chain, or the patient may be able to remove the completed test from the kit and hand-deliver or send the test to a testing provider via a shipping container package as part of the medical kit. Bar codes or other identification methods may be utilized to track the individual medical kits along the distribution channels. Patient input may also be important as an initial first step to alert the distribution system of the status of the medical kit, e.g., upon return to a kiosk, the patient scans a bar code that alerts the distribution system, clinician, and/or testing center.

With respect to the customizable medical kits, a clinician can be tied into the process somewhere along the distribution chain, where customizing is available. This may be at the regional level or the local level, or any place therebetween. The clinician may simply interact with a software program or platform that allows the clinician to pick what tools/tests the clinicians requires, and the medical kit is assembled and sent to the patient for pick-up at a designated local destination based on patient/clinician input, patient/clinician zip code or other need/information. Collection can work similar to the collection above, feeding-back to the regional distribution center for sterilization and restocking and re-use of a medical kit as needed. Again, certain tests contained in the medical kit may be removed from the kit for forwarding to a testing facility at any point during the distribution chain or the patient may be able to remove the completed test from the kit and hand deliver or send the test to a testing provider via a shipping container package as part of the medical kit. Bar codes, RFID, GPS or other identification methods may be utilized to track the individual medical kits along the distribution channels. Patient input may also be important as an initial first step to alert the distribution system of the status of the medical kit, e.g., upon return to a kiosk the patient scans a bar code that alerts the distribution system, clinician and/or testing center.

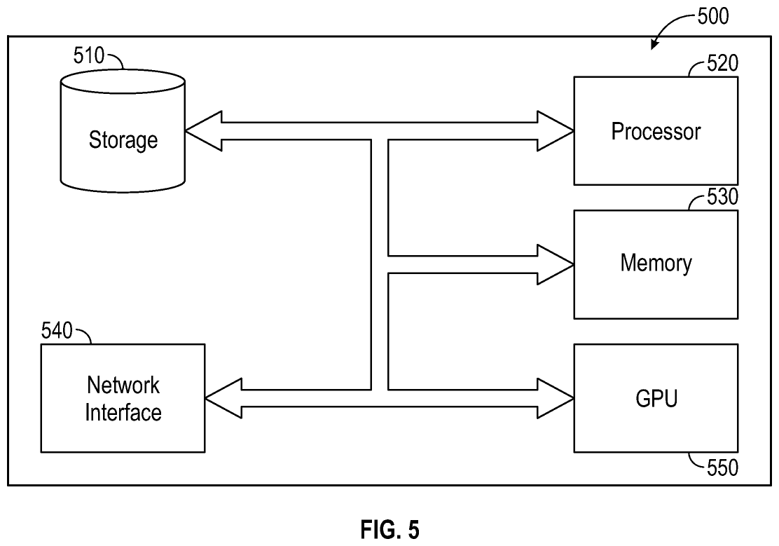

Referring now to FIG. 5, an illustration of exemplary components in the controller 500 of FIG. 1 is shown in accordance with aspects of the present disclosure. The controller 500 includes, for example, a database 510, one or more processors 520, at least one memory 530, and a network interface 540.

The database 510 can be located in storage. The term “storage” may refer to any device or material from which information may be capable of being accessed or reproduced or held in an electromagnetic or optical form for access by a computer processor. In various aspects, the controller 500 includes non-volatile memory 530 and retains stored information when it is not powered. In some aspects, the non-volatile memory includes flash memory. In certain aspects, the non-volatile memory includes dynamic random-access memory (DRAM). In some aspects, the non-volatile memory includes ferroelectric random-access memory (FRAM). In various aspects, the non-volatile memory includes phase-change random access memory (PRAM). In certain aspects, the controller 500 is a storage device including, by way of non-limiting examples, CD-ROMs, DVDs, flash memory devices, magnetic disk drives, magnetic tapes drives, optical disk drives, and cloud-computing-based storage. In various aspects, the storage and/or memory device is a combination of devices such as those disclosed herein.

In one exemplary aspect of the present disclosure, a web interface can run on the controller 500, where the interface includes a dashboard. In various aspects, data may be stored on the controller 500, including, for example, user login data, inventory, locker access date and time, and/or other data. The data can be stored in the server database 510 and sent via the system bus to the processor 520.

As will be described in more detail later herein, the processor 520 executes various processes based on instructions that can be stored in the memory 530 and utilizing the data from the database 510. With reference also to FIG. 1, a request from a user device, such as a mobile device or a client computer, can be communicated to the controller 500, through the server's network interface 540. Moreover, the disclosed structure can include any suitable mechanical, electrical, and/or chemical components for operating the disclosed systems, equipment/tools, and methods or components thereof. For instance, such electrical components can include, for example, any suitable electrical and/or electromechanical, and/or electrochemical circuitry, which may include or be coupled to one or more printed circuit boards.

As used herein, the term “controller” includes “processor,” “digital processing device” and like terms, and are used to indicate a microprocessor or central processing unit (CPU). The CPU is the electronic circuitry within a computer that carries out the instructions of a computer program by performing the basic arithmetic, logical, control and input/output (I/O) operations specified by the instructions, and by way of non-limiting examples, include server computers. In some aspects, the controller includes an operating system configured to perform executable instructions. The operating system is, for example, software, including programs and data, which manages hardware of the disclosed apparatus and provides services for execution of applications for use with the disclosed apparatus. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. In some aspects, the operating system is provided by cloud computing.

In some aspects, the term “controller” may be used to indicate a device that controls the transfer of data from a computer or computing device to a peripheral or separate device and vice versa, and/or a mechanical and/or electromechanical device (e.g., a lever, knob, etc.) that mechanically operates and/or actuates a peripheral or separate device.

In some aspects, the controller includes a display to send visual information to a user. In various aspects, the display is a cathode ray tube (CRT). In various aspects, the display is a liquid crystal display (LCD). In certain aspects, the display is a thin film transistor liquid crystal display (TFT-LCD). In aspects, the display is an organic light-emitting diode (OLED) display. In certain aspects, an OLED display is a passive-matrix OLED (PMOLED) or active-matrix OLED (AMOLED) display. In aspects, the display is a plasma display. In certain aspects, the display is a video projector. In various aspects, the display is interactive (e.g., having a touch screen or a sensor such as a camera, a 3D sensor, a LiDAR, a radar, etc.) that can detect user interactions/gestures/responses and the like. In some aspects, the display is a combination of devices such as those disclosed herein.

The controller may include or be coupled to a server and/or a network. As used herein, the term “server” includes “computer server,” “central server,” “main server,” and like terms to indicate a computer or device on a network that manages the disclosed apparatus, components thereof, and/or resources thereof. As used herein, the term “network” can include any network technology including, for instance, a cellular data network, a wired network, a fiber-optic network, a satellite network, and/or an IEEE 802.11a/b/g/n/ac wireless network, among others.

In various aspects, the controller can be coupled to a mesh network. As used herein, a “mesh network” is a network topology in which each node relays data for the network. All mesh nodes cooperate in the distribution of data in the network. It can be applied to both wired and wireless networks. Wireless mesh networks can be considered a type of “Wireless ad hoc” network. Thus, wireless mesh networks are closely related to Mobile ad hoc networks (MANETs). Although MANETs are not restricted to a specific mesh network topology, wireless ad hoc networks or MANETs can take any form of network topology. Mesh networks can relay messages using either a flooding technique or a routing technique. With routing, the message is propagated along a path by hopping from node to node until it reaches its destination. To ensure that all its paths are available, the network must allow for continuous connections and must reconfigure itself around broken paths, using self-healing algorithms such as Shortest Path Bridging. Self-healing allows a routing-based network to operate when a node breaks down or when a connection becomes unreliable. As a result, the network is typically quite reliable, as there is often more than one path between a source and a destination in the network. This concept can also apply to wired networks and to software interaction. A mesh network whose nodes are all connected to each other is a fully connected network.

In some aspects, the controller 500 may include one or more modules. As used herein, the term “module” and like terms are used to indicate a self-contained hardware component of the central server, which in turn includes software modules. In software, a module is a part of a program. Programs are composed of one or more independently developed modules that are not combined until the program is linked. A single module can contain one or several routines or sections of programs that perform a particular task.

As used herein, the controller 500 includes software modules for managing various aspects and functions of the disclosed systems or components thereof.

The disclosed systems and methods may also utilize one or more controllers 500 to receive various information and transform the received information to generate an output. The controller may include any type of computing device, computational circuit, or any type of processor or processing circuit capable of executing a series of instructions that are stored in memory. The controller may include multiple processors and/or multicore central processing units (CPUs) and may include any type of processor, such as a microprocessor, digital signal processor, microcontroller, programmable logic device (PLD), field programmable gate array (FPGA), or the like. The controller may also include a memory to store data and/or instructions that, when executed by the one or more processors, cause the one or more processors to perform one or more methods and/or algorithms.

Any of the herein described methods, programs, algorithms, or codes may be converted to, or expressed in, a programming language or computer program. The terms “programming language” and “computer program,” as used herein, each include any language used to specify instructions to a computer and include (but is not limited to) the following languages and their derivatives: Assembler, Basic, Batch files, BCPL, C, C+, C++, Delphi, Fortran, Java, JavaScript, machine code, operating system command languages, Pascal, Perl, PL1, scripting languages, Visual Basic, metalanguages which themselves specify programs, and all first, second, third, fourth, fifth, or further generation computer languages. Also included are database and other data schemas and any other meta-languages. No distinction is made between languages which are interpreted, compiled, or use both compiled and interpreted approaches. No distinction is made between compiled and source versions of a program. Thus, reference to a program, where the programming language could exist in more than one state (such as source, compiled, object, or linked) is a reference to any and all such states. Reference to a program may encompass the actual instructions and/or the intent of those instructions.

Further still, the disclosed systems and methods can utilize any number or type of analytics and/or logic approaches such as machine learning to help improve supply of the disclosed medical supply containers 400 and/or medical equipment to users. Indeed, the disclosed systems may monitor status of one or more components (e.g., storage centers, storage assemblies 100, 200, medical supply containers 400, medical equipment, etc.) and/or users and/or providers using at least one machine learning algorithm.

The terms “machine learning,” “data models,” or “artificial intelligence” may include, but are not limited to, neural networks, deep neural networks, recurrent neural networks (RNN), generative adversarial networks (GAN), Bayesian Regression, Naive Bayes, Monte Carlo Methods, nearest neighbors, least squares, means, and support vector regression, among other data science, artificial intelligence, and machine learning techniques. Exemplary uses are identifying patterns and making predictions relating to the delivery or status of the disclosed storage assemblies, medical supply containers 400, and/or medical equipment, and/or to any providers and/or users, etc. thereof.

In various aspects, the neural network may include a temporal convolutional network or a feed-forward network. In various aspects, the neural network may be trained using one or more of measuring sensor data or identifying patterns in data (e.g., increased/decreased, or anticipated supply and/or demand at various locations, for example, due to pandemic or endemic diseases, viruses, health conditions, etc. and/or changes in population, demographics, weather, etc.). In various aspects, training the machine learning algorithm may be performed by a computing device outside of the system (e.g., a remote computing device or network), and the resulting algorithm may be communicated to the controller of the system.

The term “application” may include a computer program designed to perform particular functions, tasks, or activities for the benefit of a user. Application may refer to, for example, software running locally or remotely, as a standalone program or in a web browser, or other software which would be understood by one skilled in the art to be an application. An application may run on the disclosed controllers or on a user device, including for example, on a mobile device, an IoT device, or a server system.

In one aspect of the present disclosure, the algorithms in the present disclosure may be trained. using supervised learning. Supervised learning is the machine learning task of learning a function that maps an input to an output based on example input--output pairs. It infers a function from labeled training data consisting of a set of training examples. In supervised learning, each example is a pair consisting of an input object (typically a vector) and a desired output value (also called the supervisory signal). A supervised learning algorithm analyzes the training data and produces an inferred function, which can be used for mapping new examples. In various aspects, the algorithm may correctly determine the class labels for unseen instances. This requires the learning algorithm to generalize from the training data to unseen situations in a “reasonable” way.

In various aspects, the neural network may be trained using training data, which may include, for example, different component characteristics (e.g., number of uses or changes in conditions thereof) or changes in supply and/or demand at various locations. The algorithm may analyze this training data and produce an inferred function that may allow the algorithm to identify component failures or changes in health, based on the generalizations the algorithm has developed from the training data. In various aspects, training may include at least one of supervised training, unsupervised training, or reinforcement learning.

In various aspects, the neural network may include, for example, a three-layer temporal convolutional network with residual connections, where each layer may include three parallel convolutions, where the number of kernels and dilations increase from bottom to top, and where the number of convolutional filters increases from bottom to top. It is contemplated that a higher or lower number of layers may be used. It is contemplated that a higher or lower number of kernels and dilations may also be used.

As can be appreciated, securement of any of the components of the disclosed apparatus can be effectuated using known securement techniques such welding, crimping, gluing, fastening, etc.

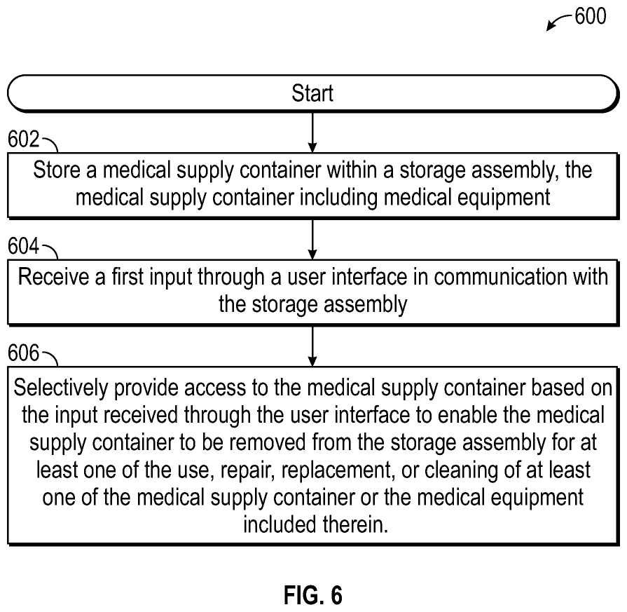

With reference to FIG. 6, a flow diagram for a computer-implemented method for supplying patients with medical equipment for remote medical interaction with healthcare providers is shown. The flow diagrams include various blocks described in an ordered sequence. However, those skilled in the art will appreciate that one or more blocks of the flow diagram may be performed in a different order, repeated, and/or omitted without departing from the scope of the disclosure. The below description of the flow diagram refers to various actions or tasks performed by one or more controller 500, but those skilled in the art will appreciate that the controller 500 is exemplary. In various embodiments, the disclosed operations can be performed by another component, device, or system. In various embodiments, the controller 500 or other component/device performs the actions or tasks via one or more software applications executing on a processor. In various embodiments, at least some of the operations can be implemented by firmware, programmable logic devices, and/or hardware circuitry. Other implementations are contemplated to be within the scope of the disclosure.

Initially, at step 602, the method stores a medical supply container within a storage assembly. The medical supply container may include medical equipment. The storage assembly may be maintained, for example, in a pharmacy, in a building having a pharmacy, in a curtilage of the pharmacy, and/or in a curtilage of the building with the pharmacy. The medical supply container with a blood pressure cuff, a pulse oximeter, an electronic stethoscope, a smart device, a thermometer, and an otoscope.

Next, at step 604, the method receives a first input through a user interface in communication with the storage assembly. For example, the first user input may include scanning a bar code and/or entering a pin number. In aspects, the method may include a platform (e.g., a webpage) for online ordering of medical supply containers 400 (FIG. 4) or boxes that selectively secure or lock medical supplies therein. For example, the medical supply container 400 may be ordered online or at a storage assembly (e.g., a kiosk) in a pharmacy.

Next, at step 606, the method selectively provides access to the medical supply container based on the input received through the user interface. For example, the user can remove the medical supply container from the storage assembly for the use, repair, replacement, and/or cleaning of the medical supply container and/or the medical equipment included therein.

In aspects, the method may further include selectively receiving the medical supply container in the storage assembly after the medical supply container is removed from the storage assembly. Thus, when the medical personnel has finished using the medical supply container, the storage assembly can selectively receive the medical supply container.

In aspects, the method may further include preventing access to the medical supply container when the medical supply container is supported in the storage assembly unless the first input includes predetermined information, such as a predetermined time to unlock the storage assembly.

In aspects, the method may further include receiving a second input through the user interface when the medical supply container is received in the storage assembly. For example, the second input indicating that the medical supply container was returned. In aspects, the method may determine a status of the medical supply container and/or the medical equipment thereof based on the second input. The method may store the status on a storage device and/or communicating the status to a server or users in communication with the server.

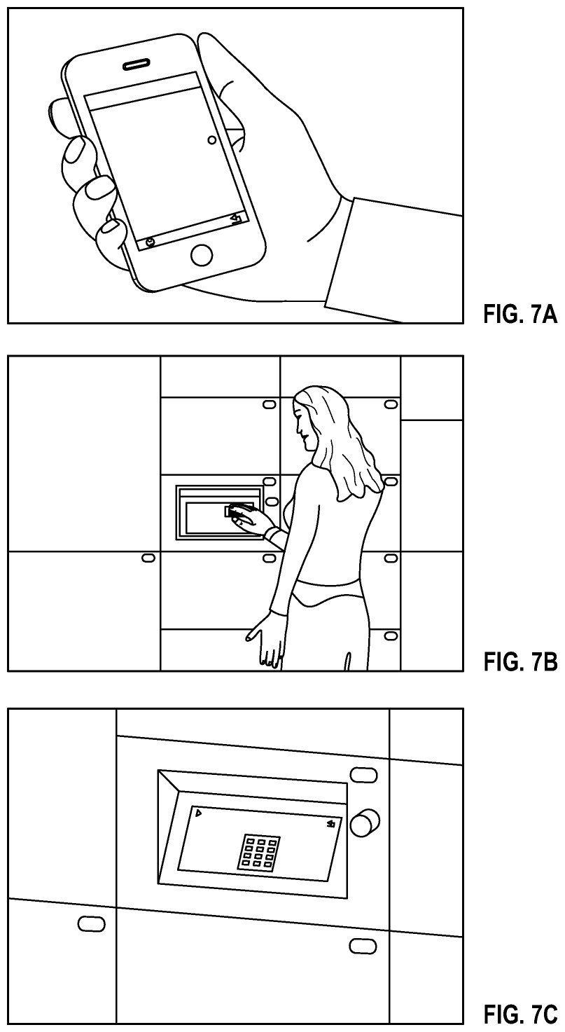

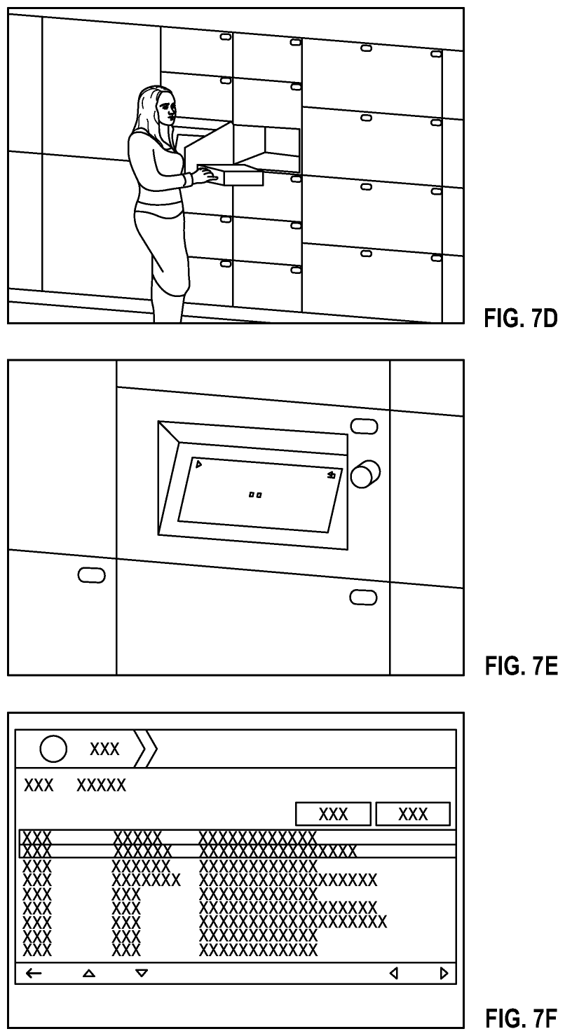

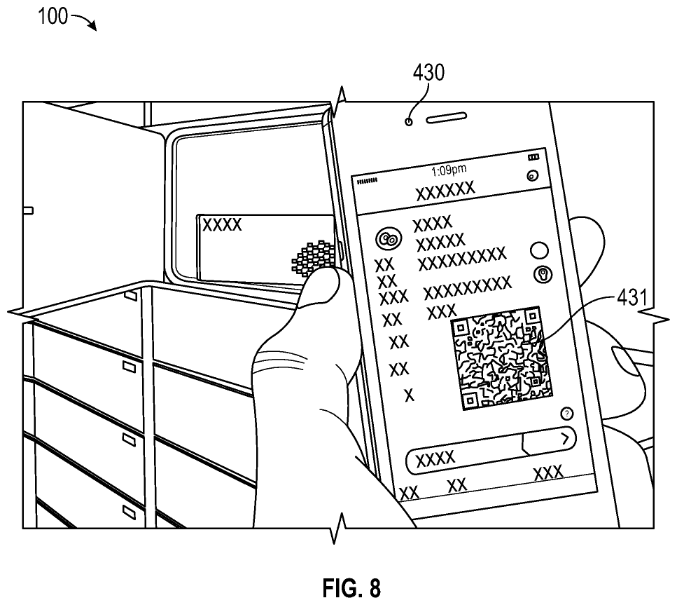

FIGS. 7A-7F and 8 are progressive views illustrating a medical supply container of the system for supplying patients with medical equipment for remote medical interaction with medical personnel being retrieved from a storage unit of the system.

FIG. 7A shows an email notification on a mobile device of a user (e.g., medical personnel). For example, the email may indicate that the medical supply container 400 (FIG. 4) is ready for pick-up. Of course, text messages or the like (e.g., social media) can be utilized for providing such notification. Next in FIG. 7B, the medical personnel goes to the storage assembly 100, 200 (e.g., kiosks, storage lockers, or smart storage lockers). Next, in FIG. 7C, the medical personnel may enter a pin or scan a bar code 431 that is emailed/texted, etc. to them (FIG. 8) for access on their mobile device 430 or other computing device, for instance.

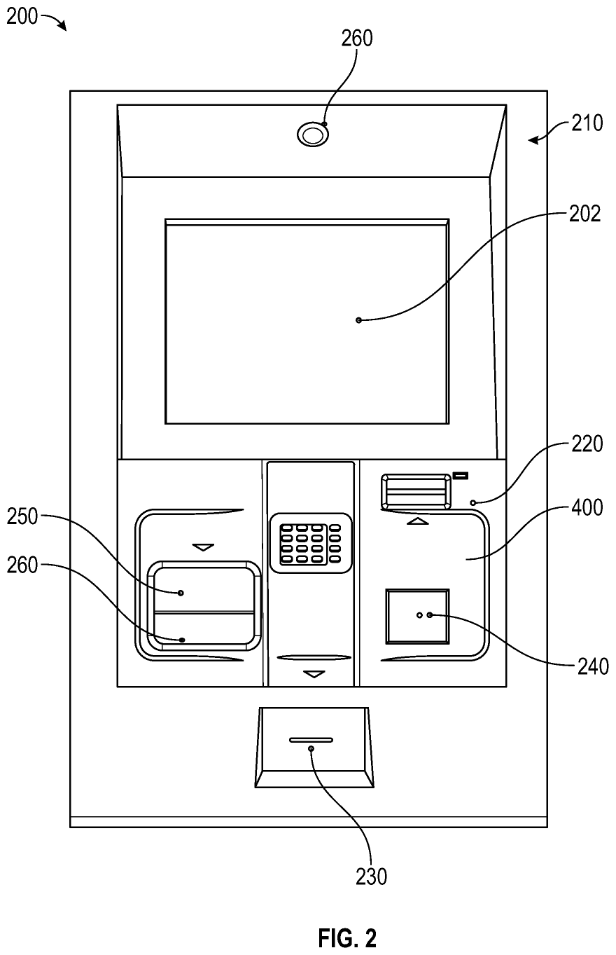

Next, a designated locker door of the storage assembly 100, 200 (FIGS. 1 and 2) open, and the medical personnel receives their medical supply container 400 and closes the locker door (FIG. 7D). FIG. 7E shows the storage assembly 100, 200 confirming the pick-up of the medical supply container 400. FIG. 7F shows an administrator receiving a report sent via email/text (or, for example, via a dashboard) showing that the medical supply container 400 has been received by the medical personnel.

Persons skilled in the art will understand that the structures and methods specifically described herein and illustrated in the accompanying figures are non-limiting exemplary aspects and that the description, disclosure, and figures should be construed merely as exemplary of particular aspects. It is to be understood, therefore, that this disclosure is not limited to the precise aspects described and that various other changes and modifications may be effectuated by one skilled in the art without departing from the scope or spirit of the disclosure. Additionally, it is envisioned that the elements and features illustrated or described in connection with one exemplary aspect may be combined with the elements and features of another without departing from the scope of this disclosure and that such modifications and variations are also intended to be included within the scope of this disclosure. Indeed, any combination of any of the disclosed elements and features is within the scope of this disclosure. Accordingly, the subject matter of this disclosure is not to be limited by what has been particularly shown and described.

What is claimed is:

1. A computer-implemented method for supplying patients with medical equipment for remote medical interaction with healthcare providers, the method comprising:
storing a medical supply container within a storage assembly, the medical supply container including medical equipment;
receiving a first input through a user interface in communication with the storage assembly; and
selectively providing access to the medical supply container based on the input received through the user interface to enable the medical supply container to be removed from the storage assembly for at least one of the use, repair, replacement, or cleaning of at least one of the medical supply container or the medical equipment included therein.

2. The computer-implemented method of claim 1, further comprising selectively receiving the medical supply container in the storage assembly after the medical supply container is removed from the storage assembly.

3. The computer-implemented method of claim 1, further comprising preventing access to the medical supply container when the medical supply container is supported in the storage assembly unless the first input includes predetermined information.

4. The computer-implemented method of claim 1, further comprising preventing access to a storage area in the storage assembly that supports the medical supply container after the medical supply container is removed from the storage area.

5. The computer-implemented method of claim 1, further comprising receiving a second input through the user interface when the medical supply container is received in the storage assembly, the second input indicating at least one of the following: that the medical supply container was returned, the medical equipment was returned, that the medical supply container was opened, the medical equipment was opened, that the medical supply container was not opened, the medical equipment was not opened, that the medical supply container was used, the medical equipment was used, that the medical supply container requires cleaning, the medical equipment requires cleaning, that the medical supply container requires replacement, the medical equipment requires replacement, that the medical supply container is ready for use, the medical equipment is ready for use, the medical equipment requires repair, or the medical supply container requires repair.

6. The computer-implemented method of claim 1, further comprising supplying the medical supply container with a blood pressure cuff, a pulse oximeter, an electronic stethoscope, a smart device, a thermometer, and an otoscope.

7. The computer-implemented method of claim 6, wherein the smart device includes a smart phone or a tablet.

8. The computer-implemented method of claim 1, further comprising receiving a code through the user interface to identify at least one of the medical supply container or the medical equipment thereof.

9. The computer-implemented method of claim 5, further comprising determining a status of at least one of the medical supply container or the medical equipment thereof based on the second input.

10. The computer-implemented method of claim 9, further comprising at least one of storing the status on a storage device or communicating the status to a server or users in communication with the server.

11. The computer-implemented method of claim 1, further comprising maintaining the storage assembly in a pharmacy, in a building having a pharmacy, in a curtilage of the pharmacy, or in a curtilage of the building with the pharmacy.

12. The computer-implemented method of claim 1, further comprising storing a plurality of medical supply containers in the storage assembly.

13. A system for supplying patients with medical equipment for remote medical interaction with healthcare providers, the system comprising:
a storage assembly that supports at least one medical supply container;
a processor; and
a memory having instructions stored thereon, which when executed by the processor, cause the system to:
receive a first request from a user interface for the at least one medical supply container;
determine if the first request meets a first predetermined criteria; and
provide access to the at least one medical supply container if the first predetermined criteria is met.

14. The system of claim 13, wherein the instructions, when executed by the processor, cause the system to:
receive a second request from the user interface to return the at least one medical supply container;
determine if the second request meets second predetermined criteria; and
if the second predetermined criteria is met, provides access to a storage area within the
storage assembly to return the at least one medical supply container.

15. The system of claim 13, wherein the instructions, when executed by the processor, cause the system to:
prevent access to the at least one medical supply container if the first predetermined criteria is not met.

16. The system of claim 13, further comprising a server in electrical communication with at least one of the storage assembly, the medical supply container, or the medical equipment.

17. The system of claim 16, wherein the server is configured to communicate with a user.

18. The system of claim 17, wherein the user includes at least one of a patient, a healthcare provider, a service provider, a vendor, a pharmacy, or a hospital.

19. The system of claim 13, wherein the memory includes a database having information stored thereon about at least one of the storage assembly, the medical supply container, the medical equipment, patients, healthcare providers, service providers, vendors, pharmacies, or hospitals.

20. A system for supplying patients with medical equipment for remote medical interaction with healthcare providers, the system comprising:
a plurality of storage assemblies supporting a plurality of medical supply containers; and
a server in electrical communication with at least one of the plurality of storage assemblies or the plurality of medical supply containers, the server configured to receive a request through a user interface for temporarily providing access to a first medical supply container of the plurality of medical supply containers.

21. The system of claim 20, wherein the server is configured to receive a request through the user interface for returning the medical supply container to one of the plurality of storage assemblies.

22. The system of claim 20, wherein the server is configured to communicate with a user.

23. The system of claim 20, wherein the user includes at least one of a patient, a healthcare provider, a service provider, a vendor, a pharmacy, or a hospital.

24. The system of claim 23, wherein healthcare providers include at least one of doctors, dentists, nurses, physician assistants, therapists, pharmacists, trainers, nutritionists, or other healthcare personnel.

25. The system of claim 23, wherein the service provider includes a repair service provider, a cleaning service provider, or an equipment supplier.

26. The system of claim 20, wherein the server includes at least one of input or output modules for transmitting and receiving at least one of input or output signals between at least one of components of the system or users of the system.
